(19) United States
(12) Patent Application Publication
(10) Pub. No.: US 2012/0016404 A1
(43) Pub. Date: Jan. 19, 2012

(54) VERTEBRA FILLER

(76) Inventor: Weixing Shao, Shandong (CN)

(21) Appl. No.: 13/257,599

(22) PCT Filed: Jun. 5, 2009
(86) PCT No.: PCT/CN2009/000626

§ 371 Date: Sep. 20, 2011

(51) Int. Cl. A61M 29/00

(52) U.S. Cl. 606/192

(57) ABSTRACT

The present invention offers two programs of a filler for spine, both two include a catheter and a catheter holder for fastening the catheter, there is a guide pin inside of the catheter, and the guide pin is fastened on the guide pin holder, a sealed or ventilate feed bag made of soft nonmetal materials accordent to biocompartibility or absorbefacient materials is set at the end of the catheter, the feature of the two programs lies in that a outer thimble is set at the outerside of the catheter, the outer thimble is fixed on the outer thimble holder, a dismountable structure connects the outer thimble holder and catheter holder, a reducing cone of tapered outer thimble at the open end of the said outer thimble connects to a little diameter outer thimble, or a inside convex plate of outer thimble; the said catheter has a reducing cone corresponding to the reducing cone of outer thimble at the reducing cone of outer thimble or the inside convex plate of outer thimble, and the reducing cone of catheter connects to a little diameter catheter; the said feed bag is set in the catheter through its open end, lies in between outer thimble and catheter and fastened tightly at the outer wall of catheter by outer thimble.

### BACKGROUND OF THE INVENTION

The present invention relates to a kind of medical device, especially a filler for spine which is suit for comminuted fracture.

The widely-known technology is NO.200820173700.0 patent applied by myself in state intellectual property office in the existing technologies, the patent discloses a spreader and filler for surgery of compression fracture, this filler includes catheter and cather holder for fastening catheter, there is a guilde pin inside of the catheter, and the guide pin is fixed on the guide pin holder, a feed bag is set at one end of the catheter, the joint of the said feed bad connects to the catheter with thread, revolving catheter must be done for the separation of feed bag and catheter of this kind of connection after surgery finished, that increases the difficulty of the surgery, in addition, the joint should be left in the body. Those are the shortcomings of the existing technologies.

### BRIEF SUMMARY OF THE INVENTION

The purpose of the present invention is to offer two technical programs of a filler for spine aiming at the shortcomings of existing technologies. The feed bag is set at the outer wall of the catheter by outer thimble in the programs, which is convenient for surgery, when the surgery finished, the catheter and outer thimble could be drawn out and useless foreign matter need not leave in the body.

The program is achieved through the following technical measures:

The filler for spine in the first program includes a catheter and a catheter holder for fastening the catheter, there is a guide pin inside of the catheter, and the guide pin is fastened on the guide pin holder, a sealed or ventilate feed bag made of soft nonmetal materials accordent to biocompartibility or absorbefacient materials is set at the end of the catheter, the feature of the program lies in that a outer thimble is set at the outerside of the catheter, the outer thimble is fixed on the outer thimble holder, a dismountable structure connects the outer thimble holder with the catheter holder, a reducing cone of tapered outer thimble at the open end of the said outer thimble connects to a little diameter outer thimble; the said catheter has a reducing cone corresponding to reducing cone of outer thimble at the reducing cone of outer thimble, and the reducing cone of catheter connects to a little diameter catheter; the said feed bag is set in the catheter through its open end, lies in between outer thimble and catheter and fastened tightly at the outer wall of catheter by outer thimble. The specific feature of the program also includes: the length of the said little diameter catheter is 1-40 mm, that of the said little diameter outer thimble is 0-10 mm, and that of the said feed bag is between 10-50 mm. The said feed bag is a monolayer, and a protective layer for feed bag is at the outer wall of the said feed bag, the dismountable connection between the said outer thimble holder and catheter holder is achieved through a thread.

The filler for spine in the second program includes a catheter and a catheter holder for fastening the catheter, there is a guide pin inside of the catheter, and the guide pin is fastened on the guide pin holder, a sealed or ventilate feed bag made of soft nonmetal materials accordent to biocompartibility or absorbefacient materials is set at the end of the catheter, the feature of the program lies in that a outer thimble is set at the outerside of the catheter, the outer thimble is fixed on the outer thimble holder, a dismountable structure connects the outer thimble holder with catheter holder, a inside convex plate is set at the open end of outer thimble; the said catheter has a reducing cone corresponding to inside convex plate of outer thimble at the inside convex plate of outer thimble, and the reducing cone of catheter connects to a little diameter catheter; the said feed bag is set in the catheter through its open end, lies in between outer thimble and catheter and fastened tightly at the outer wall of catheter by outer thimble. Other specific features of the program includes: the length of the said little diameter catheter is 1-40 mm, that of the said little diameter outer thimble is 0.5-10 mm, and that of the said feed bag is between 10-50 mm. The said feed bag is a monolayer, and a protective layer for feed bag is at the outer wall of the said feed bag, the dismountable connection between the said outer thimble holder and catheter holder is achieved through a thread.

The beneficial effects of the programs can be recognized by the above narration, in the two structures, the said feed bag is set at the outer wall of the catheter by outer thimble, so the born materials such as ferroconcrete can be injected into the feed bag by catheter to support the spine of the patient. When the surgery finished, the catheter and outer thimble could be drawn out and useless foreign matter need not leave in the body. The difference of the two programs only lies in the structure limits the feed bag not to slip freely. In the first program, the structure adopted is a reducing cone of a tapered outer thimble connects to a little diameter outer thimble at the open end of the outer thimble, and the catheter has a reducing cone corresponding to that of outer thimble at the reducing cone of outer thimble, and the reducing cone of catheter connects to a little diameter catheter. In the second program, the structure adopted is a inside convex plate at the open end of the outer thimble, and the catheter has a reducing cone corresponding to the inside convex plate at the inside convex plate of outer thimble, and reducing cone of catheter connects a little diameter catheter. The two programs adopt the changing diameter of feed bag when moving, to limit the feed bag not to move freely. Thus, the present invention has substantive features and progress compared with existing technologies, it is also obvious for its beneficial effects of implementation.

### BRIEF DESCRIPTION OF THE DRAWINGS

FIG. 1 is the partly sectional structure view of embodiment of the invention.

Where, 1 is feed bag, 2 is guide pin, 3 is little diameter catheter, 4 is little outer thimble, 5 is reducing cone of outer thimble, 6 is reducing cone of catheter, 7 is catheter, 8 is outer thimble, 9 is outer thimble holder, 10 is catheter holder, 11 is guide pin holder, 12 is inside convex plate of outer thimble.

### DETAILED DESCRIPTION OF THE INVENTION

In order to clearly explain the technical features of the program, the following will describe the programs by two embodiments and their drawings.

### The First Embodiment

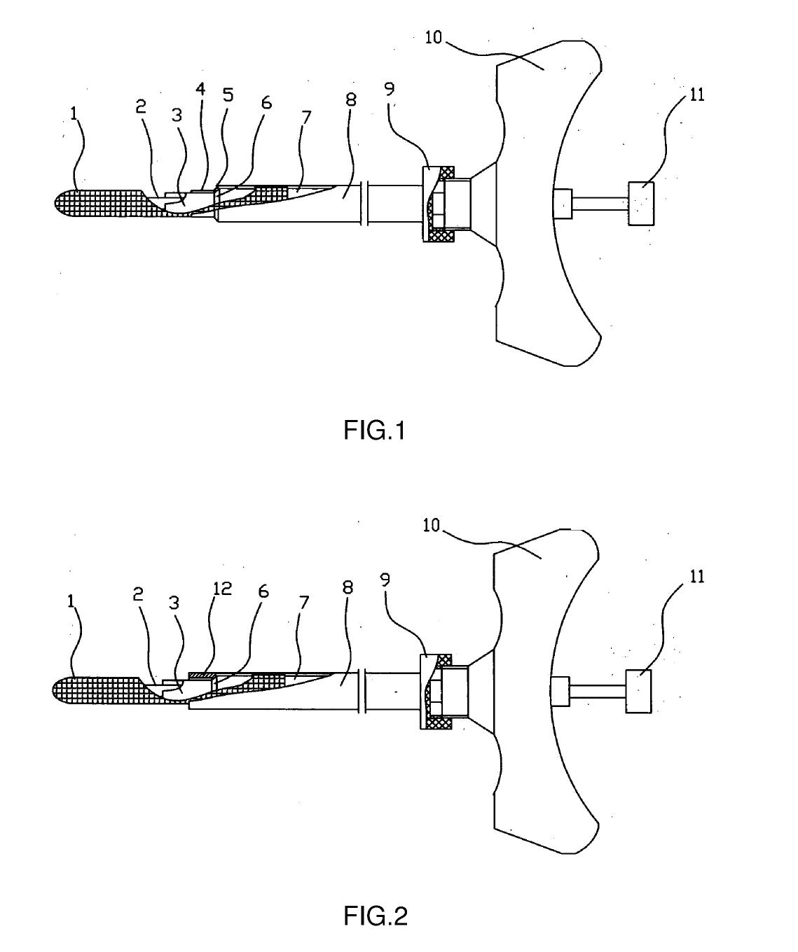

It can be seen from the FIG. 1, the filler for spine of the embodiment includes a catheter 7 and a catheter holder 10 for fastening catheter 7. There is a guide pin 2 inside of the catheter 7, and the guide pin 2 is fastened on the guide pin holder 11, a sealed or ventilate feed bag 1 made of soft nonmetal materials accordent to biocompartibility or absorbefacient materials is set at the end of the catheter 2, a outer thimble 8 is set at the outerside of the said catheter 7 in this embodiment, and the outer thimble 8 is fixed on the outer thimble holder 9, a dismountable structure connects the outer thimble holder 9 and catheter holder 10, the thread connection is adopted in this embodiment. a reducing cone of tapered outer thimble 5 at the open end of the said outer thimble 8 connects to a little diameter outer thimble 4; the length of the little diameter outer thimble 4 is 0-10 mm; the said catheter 7 has a reducing cone of catheter 6 corresponding to reducing cone of outer thimble 5 at the reducing cone of outer thimble 5, and the reducing cone of catheter 6 connects to a little diameter catheter 3, the length of the little diameter catheter 3 is 1-40 mm; In this embodiment, the length of the little diameter outer thimble 4 is 3 mm, and that of the little diameter catheter 3 is 10 mm. The said feed bag 1 is set in the catheter 7 through its open end, lies in between outer thimble 8 and catheter 7 and fastened tightly at the outer wall of catheter 7 by outer thimble 8. The length of the feed bag 1 is between 10-50 mm. The said feed bag 1 can be a monolayer or a protective layer is added at the out wall of the feed bag 1.

### The Second Technical Embodiment

It can be seen from FIG. 2, the filler for spine in the program includes a catheter 7 and a catheter holder 10 for fastening the catheter 7, there is a guide pin 2 inside of the catheter 7, and the guide pin 2 is fastened on the guide pin holder 11, a sealed or ventilate feed bag 1 made of soft nonmetal materials accordent to biocompartibility or absorbefacient materials is set at the end of the catheter 2, a outer thimble 8 is set at the outerside of the said catheter 7 in this embodiment, the outer thimble 8 is fixed on the outer thimble holder 9, a dismountable structure connects the outer thimble holder 9 and the catheter holder 8, the thread connection is adopted in this embodiment. A inside convex plate 12 is set at the open end of outer thimble 8, the length of the inside convex plate 12 is 0.5-10 mm; the said catheter 7 has a reducing cone 6 corresponding to inside convex plate of outer thimble 12 at the inside convex plate of outer thimble 12, and the reducing cone 6 of catheter connects to a little diameter catheter 3; the length of the little catheter 3 is 1-40 mm. In this embodiment, the length of the inside convex plate 12 of outer thimble is 5 mm, and that of the little diameter of catheter 3 is 15 mm. The said feed bag 1 is set in the catheter 7 through its open end, lies in between outer thimble 8 and catheter 7 and fastened tightly at the outer wall of catheter 7 by outer thimble 8, the length of feed bag 1 is between 10-50 mm. The said feed bag 1 can be a monolayer or a protective layer is added at the out wall of the feed bag 1.

What is claimed is:

1. A filler for spine includes a catheter and a catheter holder for fastening the catheter, there is a guide pin inside of the catheter, and the guide pin is fixed on the guide pin holder, a sealed or ventilate feed bag made of soft nonmetal materials accordent to biocompartibility or absorbefacient materials is set at the end of the catheter, wherein a outer thimble is set at the outerside of the said catheter, the outer thimble is fixed on the outer thimble holder, a dismountable structure connects the outer thimble holder with catheter holder, a reducing cone of tapered outer thimble at the open end of the said outer thimble connects to a little diameter outer thimble; the said catheter has a reducing cone corresponding to the reducing cone of outer thimble at the reducing cone of outer thimble, and the reducing cone of catheter connects to a little diameter catheter; the said feed bag is set in the catheter through its open end, lies in between outer thimble and catheter and fastened tightly at the outer wall of catheter by outer thimble.

2. The filler for spine of claim 1, wherein the length of the said little diameter catheter is 1-40 mm.

3. The filler for spine of claim 1 or 2, wherein the length of the said little diameter outer thimble is 0-10 mm.

4. A filler for spine includes a catheter and a catheter holder for fastening the catheter, there is a guide pin inside of the catheter, and the guide pin is fixed on the guide pin holder, a sealed or ventilate feed bag made of soft nonmetal materials accordent to biocompartibility or absorbefacient materials is set at the end of the catheter, wherein a outer thimble is set at the outerside of the said catheter, the outer thimble is fixed on the outer thimble holder, a dismountable structure connects the outer thimble holder and catheter holder, a inside convex plate of outer thimble is set at the open end of the said outer thimble; the said catheter has a reducing cone corresponding to the inside convex plate of outer thimble at the inside convex plate of outer thimble, and the reducing cone of catheter connects to a little diameter catheter; the said feed bag is set in the catheter through its open end, lies in between outer thimble and catheter and fastened tightly at the outer wall of catheter by outer thimble.

5. The filler for spine of claim 4, wherein the length of the said little diameter catheter is 1-40 mm.

6. The filler for spine of claim 4 or 5, wherein the length of the said little diameter outer thimble is 0.5-10 mm.

7. The filler for spine of claim 1 or 4, wherein the length of the said feed bag is 10-50 mm.

8. The filler for spine of claim 1 or 4, wherein the said feed bag is a monolayer.

9. The filler for spine of claim 1 or 4, wherein the said feed bag has a protective layer at its outside wall.

10. The filler for spine of claim 1 or 4, wherein the dismountable connection between the said outer thimble holder and the said catheter holder is a thread one.
